(19) United States
(12) Patent Application Publication
(10) Pub. No.: US 2002/0007500 A1
(43) Pub. Date: Jan. 17, 2002

(54) Molecular control of transgene escape by a repressible excision system

(76) Inventors: Viktor Kuvshinov, Helsinki (FI); Kimmo Koivu, Helsinki (FI); Anne Kanerva, Helsinki (FI); Eija Pehu, Helsinki (FI)

Correspondence Address: BROWDY AND NEIMARK, P.L.L.C., 624 NINTH STREET, NW, SUITE 300, WASHINGTON, DC, 20001-5303, US

(21) Appl. No.: 09/783,292

(22) Filed: Feb. 15, 2001

Related U.S. Application Data
(63) Continuation-in-part of application No. 09/617,543, filed on Jul. 14, 2000.

(51) Int. Cl. C12N 15/82; C12N 15/87

(52) U.S. Cl. 800/278; 800/287; 800/288

(57) ABSTRACT

The present invention is related to a method and a system for controlling the transgene segregation and spread. Escape of the gene of interest (TGI) into the environment is prevented by a repressible excision system (RES), which can be controlled by externally applicable means and comprises an excision construct (EC) having a gene encoding recombinase closely linked to the (TGI) and flanked by excision recognition sites (ERSs). The externally applicable means for controlling the repression of the expression of the gene encoding the recombinase enzyme is achieved with or without a repressor construct (RC). The action of the repressible excision system (RES) leads to excision of the transgenic insert, whenever a transgenic organism and the externally applicable means are withdrawn, which occurs if a transgenic organism escapes from the human control.

### THE TECHNICAL FIELD OF THE INVENTION

The present invention is related to a method and a DNA construct comprising a repressible excision system (RES) for controlling transgene release to the environment by a mechanism, which automatically excises the transgenic insert when an externally applicaple means controlled by man is withdrawn, which happens if a transgenic organism escapes, for example by crossing with related wild type or non-transgenic species. The DNA construct responsible for the repressible excision, comprises one or more excision constructs (ECs) which are provided with one or more genes encoding a recombinase, the expression of which is repressible by using an externally applicable and/or regulated mechanism either alone or in combination with one or more repression constructs (RCs) capable of repressing the expression of said recombinase. The present invention is useful for the production of transgenic, sexually reproducing multicellular organisms (SRMOs), especially plants and certain animals, such as fish.

### THE BACKGROUND OF THE INVENTION

Over the past few years safety questions in relation to transgenic crop production have attracted a lot of attention among the scientific community and the public at large. Several reports have shown that transgenes have spread from the field plots through pollen. Most problematic are crop species, such as Brassicae, cereals or certain trees, which have wild relatives in nature and which can hybridize with the transgenic crop.

The rapid progress in molecular biology and transgenic technology will probably lead to increased use of transgenic plants. Consequently, the need to produce new methods and systems for preventing transgene escape is intensified. While the potential risks to human or animal health of a particular transgene or its gene product can be tested and measured, the impact of gene escape is more complex to assess. However, the potential of transgenic crop improvement is so tremendous, that it is probably more efficient to find solutions for preventing the gene from escaping than banning the use of transgenes.

Several attempts to solve the problem have been made. For example, the patent U.S. Pat. No. 5,723,765 discloses a method for arresting the germination and/or function of seed. A drawback of said method is that once the plants have been treated with the agent activating the killer gene, they cannot be rescued. As a consequence the use of the transgenic seed is limited to one generation, because there is no way to stop the killer gene. Another drawback is that the transgenes may escape in case the killer gene construct is not activated, because the transgenic plant in that case is still capable of germinating, growing to maturity, flowering and sexually reproduce with related wild type or non-transgenic cultivated species.

A method for controlling the segregation of transgenes and their escape by a recoverable block of function system has been disclosed by the present inventor in the patent application U.S. Ser. No. 09/617,543. Plants carrying the construct described in U.S. Ser. No. 09/617,543 will not disperse into the environment, because a function necessary for their survival and reproduction is blocked, when the transgenic seed is not subjected to a mechanism which may be externally regulated. According to said method the seeds from a transgenic crop, if shattered during harvest, will not germinate and reproduce in a normal way under the natural conditions existing in the environment. Therefore, the sexually reproducing multicellular organism, the SRMO, carrying a transgene does not survive and consequently the transgene disintegrates in nature. Furthermore, the transgenic seed disclosed in U.S. Ser. No. 09/617,543 and its use for crop production is not limited to only one generation. The customer or farmer provided with the appropriate instructions as regards the external means for reviving or recovering of the blocked function can reuse the transgenic seed. Thus, the invention disclosed in U.S. Ser. No. 09/617,543 allows controlled cultivation and use of transgenic plants, but prevents their survival and reproduction, in case they escape into the environment.

In the present invention, the inventor of the method disclosed in U.S. Ser. No. 09/617,543 presents an alternative solution including methods and DNA constructs for preventing transgenes from escaping. In the present invention, instead of blocking a function which is essential for the survival or reproduction of a transgenic organism or plant, externally applicaple means are used to control a repressible excision system (RES) which controls the expression and/or repression of a gene encoding a recombinase enzyme. In other words, the repressible excision system (RES) responds to an outside stimulus and thereby controls the excision of the transgenic insert. The repressible excision system (RES) comprises one or more excision constructs (ECs) carrying one or more genes encoding a recombinase enzyme capable of excising a transgenic insert flanked by appropriate excision recognition sites (ERSs).

### THE SUMMARY OF THE INVENTION

The characteristic features of the present invention are as defined in the claims. More specifically, the method of the present invention for controlling the transgene segregation in a sexually reproducing multicellular organism (SRMO) and for preventing the escape of said transgene into the environment is based on a molecular mechanism which herein is called a repressible excision system (RES), which comprises in addition to one or more genes of interest (TGIs) encoding desired gene products, at least one excision construct (EC) comprising a gene encoding a recombinase, i.e. an enzyme, capable of excising a transgenic insert, said insert being flanked by at least two excision recognition sites (ERSs). Further, the repressible excision system (RES) comprises at least one means for controlling the recombinase expression and/or repression, respectively. Said means are either externally applicaple means, such as heat, light, osmotic and/or chemical supplements. Said externally applicable means can control the expression and/or repression either directly or indirectly in combination with so called repression construct(s) (RC).

The gene encoding recombinase placed in the excision construct (EC), may comprise operably linked sequences capable of controlling the expression or repression, e.g. promoters, operons, signal sequences etc. The gene encoding the recombinase enzyme and its promoters and operators can be controlled or regulated by one or more separate repression constructs (RCs), which also can be controlled by externally applicable means. One or more of said repression constructs (RCs) are preferably used for controlling the expression of the recombinase enzyme. But in the broadest aspect of the present invention the repression construct (RC) is optional, meaning that the excision construct (EC) responds directly to the outside stimulus or human treatment. The repression constructs (RCs) can be placed either in the same chromosome in the same insert or locus as the excision construct (EC) and the transgene of interest (TGI) or in different non-allelic chromosomes. Depending upon the sites and numbers of the excision and/or repression constructs (EC and RC), the disappearance of the transgenic insert differs or takes place in different order or in different time intervals as disclosed in the description of segregation occuring after the hybridization. Depending on the number and sites of excision and/or repression constructs (ECs and RCs) several types of repressible excision systems (RESs) can be recognized. These include general, delayed, reversed delayed, double, triple and multiple as well as combined or mixed repressible excision systems (RES). The mixed systems include constructs of the block of function (RBF) systems. Said repressible excision systems (RESs) and their functions are described in more detail below.

The nucleotide sequence or excision construct (EC) encoding the recombinase enzyme is generally placed in the same locus as the transgenic insert or the transgene of interest (TGI). In the present invention it is essential that the means for repressing the excision construct (EC), i.e. the genes encoding the recombinase enzyme comprise at least one externally applicable means of control. This means that the repression can be regulated by man. The externally applicable means is for example an artificial manipulation step which can be used to control one or more repressing constructs (RCs) or the function facilitating sequences, such as promoters, operons, etc., which are operably linked to the gene encoding the recombinase enzyme.

The escape of the transgene is prevented by the excision of the transgenic insert from the genome of the SRMO, because the repressible excision system (RES) is such that it automatically switches on or turns on the expression of the recombinase enzyme if the externally applicable, artificial, manipulation step or treatment is withdrawn. This happens for example when a transgenic organism hybridizes with a related wild-type or non-transgenic cultivated organism. When recombinase expression starts as a result of withdrawal of the externally applicable means, the expressed recombinase enzyme recognizes the signal sites, excision recognition sites (ERSs) flanking the transgenic insert and excises the transgenic insert, which thereafter disintegrates in the cell.

The expression of the recombinase gene, ultimately meaning the excision of the transgenic insert, must be repressed in order to allow an undisturbed expression of the transgenes of interest (TGIs). Otherwise the production of the desired product of the transgene of interest (TGI) is not facilitated. The repression is achieved by applying at least one externally applicable, artificial means or manipulation step, which can be controlled by man. The manipulation step may be applied in the form of alternative or combined physical or chemical means, e.g. temperature, light, osmotic and hormone supplements, etc.

The transgenic insert including the transgene of interest (TGI) is automatically controlled during interbreeding and outbreeding of the SRMO with closely related, either cultivated or wild species, because the artificial manipulation step applied from outside is automatically removed when the transgenic SRMO escapes into the environment. In other words, as soon as no man-controlled treatment is applied, the repression mechanism fades away, loss of recombinase repression occurs and the transgenic insert in the genome of the sexually reproducing multicellular organism (SRMO) is excised and disintegrates. Consequently, the transgenic insert in the SRMO outside human control, automatically disappears and is automatically prevented from leaking into the environment.

The expression of the recombinase enzyme, for which the excision construct (EC) in the host organism controls or is responsible for, is either constitutive or development or organ specific in nature and must furthermore be repressible. If the expression of recombinase is constitutive it is essential that the repression construct (RC) responds either to an outside controllable stimulus or provides a continuous repression in case the repression construct (RC) is placed in a different non-allelic chromosome.

In the method of the present invention the action of the gene encoding the recombinase enzyme causes the excision of those DNA sequences, which are flanked on both sides, by specific signal sequences, herein defined as excision recognition sites (ERSs).

The repression of the expression of the gene encoding the recombinase enzyme is as said above either constitutive or inducible in an organ specific and developmental manner. In both cases, it is necessary that the expression and/or repression should respond to an external stimulus. The constitutive expression of recombinase also requires a continuous repression. The repression of the expression of the recombinase enzyme is achieved by various actions, which preferably are selected from a group consisting of blocking the action of a promoter, expressing antisense RNA of recombinase and/or expressing a DNA binding protein.

The promoter preferably comprises an operator sequence introduced into the promoter sequence of the gene encoding the recombinase enzyme. The gene or construct responsible for the repression may express a repressor protein having the capacity of binding to an operator sequence introduced into the promoter of the recombinase. Thereby it represses the recombinase expression.

The externally regulated molecular mechanism repressing the recombinase enzyme includes at least one externally applicable manipulation step. Several of these are discussed below. It is for example possible to add one or more externally applicable substances. Such substances are for example certain sugars, which are necessary for activating certain inducible promoters or alternatively repress the promoter. One or more external chemical or physical stimuli, which have the capacity of activating the repression mechanism are advantageously used for controlling or regulating the repression. These stimuli include heat, light, osmosis etc. It is also possible to repress the promoter of the gene expressing recombinase by the activity of another DNA construct e.g. a repression construct (RC) which comprises nucleotide sequences expressing binding proteins or antisense RNA. Antisense RNA technology may advantageously be used for silencing the recombinase RNA expression. Another alternative for repressing is to express a protein, which specifically binds to a nucleotide or amino acid sequence which controls the repression of recombinase expression.

The gene encoding the recombinase enzyme is preferably placed in the genome of SRMO in the same transgenic insert with the transgene of interest (TGI). It can be also placed between two transgenes of interest (TGI) or in a sufficiently long intron of the transgene of interest (TGI). The gene encoding the recombinase enzyme may also be placed in a complex system for controlling the escape of the transgene of interest (TGI). The complex system for controlling the escape of transgene of interest (TGI) can be a reversed, double, triple or a multiple repressible excision system (RES), optionally combined with one or more recoverable block of function (RBF) systems described in U.S. Ser. No. 09/617,543. Said complex or mixed systems, which are evident for one skilled in the art reading this specification and that disclosed in U.S. Ser. No. 09/617,543 enable preparation of innumerable variations and/or different system for controlling the escape of transgenes.

When the excision construct (EC) including the gene(s) encoding recombinase and the sequences controlling the function of said gene, which sequences are operably linked to the gene encoding the recombinase enzyme and the repression construct (RC) including the repressor gene(s) capable of controlling the repression by responding to outside stimuli are placed in the same chromosome they result in a so called general repressible excision system (RES), which is described in more detail below. When the expression construct (EC) including the recombinase gene and the repression construct (RC) including gene(s) are placed in different non-allelic chromosomes they result in a delayed repressible excision system (RES) also described in more detail below.

The DNA constructs of the present invention, i.e. the repressible excision system (RES) for controlling transgene segregation and for preventing transgenes from escaping into the environment comprise at least one gene expressing a recombinase enzyme, i.e. a gene expressing a substance or an enzyme capable of excising the transgenic insert. The gene encoding the recombinase enzyme and/or the expression of said gene must be repressible by externally applicable means acting directly on the gene encoding the recombinase enzyme or on other sequences optionally capable of responding to outside stimuli and thereby regulating or controlling the repression or expression of said recombinase enzyme.

The repressible excision system (RES) of the present invention preferably includes one or more externally regulated repression construct(s) (RCs), but systems acting without said repression constructs (RCs) can be constructed. The repressible excision system (RES) with its DNA construct(s) comprise(s) also at least one gene encoding a recombinase enzyme and at least two excision recognition sites (ERSs) i.e. (DNA sequences) flanking the transgenic insert.

The externally regulated repressible excision system (RES) can be used for excising the transgene in a desired stage of cultivation by inactivating the repression function. For example, the transgenic insert can be excised by adding tetracycline, which removes the repression caused by a TetR repression construct (RC). The removed transgene can perform a function undesired in further cultivation or processing of the sexually reproducible multicellular organism (SRMO).

The excision construct (EC) comprising a DNA sequence responsible for the expression of a recombinase enzyme is preferably repressible by the action of the repression construct (RC). The repression of the expression of the recombinase enzyme is achieved, for example, by blocking a promoter action by binding a repressor protein or by expressing an antisense RNA of the gene encoding the recombinase. The blocking of the action of the promoter occurs at the operator sequence(s) introduced into the promoter of the recombinase gene. The repressor gene expresses the repressor protein having the ability to bind to the operator sequence(s) introduced into the promoter of the gene encoding the recombinase enzyme and by said binding it also represses the expression of the recombinase.

The complex system for controlling the escape of transgene of interest is a so called double, reversed or multiple (triple) repressible excision system (RES) combined with a recoverable block of function (RBF) system resulting in a mixed system.

### A SHORT DESCRIPTION OF THE DRAWINGS

FIG. 1 is a schematic presentation of the repressible excision system (RES). An externally regulated repression construct (RC) represses the function of the excision construct (EC) during controlled cultivation of the sexually reproducible multicellular organism (SRMO). The transgenic insert carrying transgene(s) of interest (TGI) will be excised from the genome of the SRMO when the function of the repression construct (RC) is lost and the excision construct (EC) is activated and the expression of the recombinase is initiated and results in the excision of the transgenic insert, i.e. the DNA sequences flanked by the excision recognition sites (ERSs) under natural non-controlled conditions.

FIG. 2 is a schematic presentation of molecular constructs used in the repressible excision system (RES) and described in Example 1. Boxes of promoters presented with arrowheads show the direction of the gene sequences. The transgenic insert is flanked by the LOX—recognition sites for excision (ERS). The abbreviations are p for promoter; 3′ for 3′ end of the gene (polyadenylation site).

FIG. 2a presents the detailed construct is presented to show the principles of placing the genes.

FIG. 2b illustrates the same construct on a more general level.

FIG. 3 depicts the molecular constructs described in Example 2. Boxes of promoters with arrowheads show the direction of the gene sequences. The abbreviations are p for promoter, 3′ for 3′ end of the gene (polyadenylation site), LOX for signal site for excision of DNA sequence by Cre recombinase. Boxes of promoters show the direction of the gene sequences. The the first and second constructs in chromosome I and II are shown separately.

FIG. 3a presents the construct in chromosome I is presented in detail (upper row) to show the principles of placing the genes and on a more general level (lower row).

FIG. 3b presents the construct in a second chromosome II in detail and on a more general level.

FIG. 4 shows the molecular constructs comprising a triple recoverable block of function system/repressible excision system (RBF/RES), which constructs are described in Example 3. The principle locations or preferred positions of the genes in the constructs are presented. Three constructs (transgenic inserts are placed or introduced into three different non-allelic chromosomes (I, II, and III). The recoverable block of function (RBF) system comprises barstar and barnase genes. The repressible excision system (RES) comprises cre recombinase and the repression construct (RC) comprises an antisense and a part of a sense cre coding sequence. The interaction of the recovering—blocking and repression—excision functions is shown by arrows.

FIG. 5 is a schematic drawing showing the hybridization of the transgenic plants carrying a triple construct comprising a recoverable block of function system and repressible excision system (RBF/RES). The constructs of the recoverable block of function (RBF) system are marked B and R for blocking and recovering constructs, respectively. E and P are used to mark the excision and repression constructs (EC and RC), respectively belonging to the repressible excision system (RES). The blocking (B) and recovering (R) constructs as well as the excision (E) and repression (P) constructs are placed in different non-allelic chromosomes (marked by a circle or a pentagon) in opposite order and linked in pairs B+P and E+R. The first transgene of interest (I) is placed in a third non-allelic chromosome (marked by a square). It is placed between the blocking (B) and the excision (E) constructs. The system described above comprises an introline crossing or hybridization, which supports the homozygous genotype of both recoverable block of function (RBF) system and repressible excision system (RES) in the progeny. Transgenic homozygous parental line (PL) is hybridized with another transgenic homozygous line or wild type (WT) relative plant. Said introline crossing or hybridization produces transgenic homozygous F1 hybrid progeny. Outside crossing or hybridization with a wild type (WT) produces transgenic heterozygous F1 progeny. The F1 progeny of outside hybridization carries a heterozygous genotype for both the recoverable block of function (RBF) and repressible excision system (RES) constructs and the transgene of interest (I). The progeny provides plants which are capable of reproducing and which carry all transgenic inserts. The hybridization of heterozygous F1 progeny with WT produces F2 progeny. Segregation of the constructs in F2 progeny of outside hybridization leads to a strong negative selection of the transgene of interest (I). Only one of eight hybrid genotypes is capable of sexual reproduction and carries the transgene of interest (TGI). None of the transgenic constructs can freely segregate into non-transgenic relative genomes. Slashed genotypes are suicidal in the result of action of the blocking construct (BC). Slashed transgenic inserts are excised from the genome of SRMO in the result of action of excision construct (EC).

FIG. 6 depicts the molecular constructs described in Example 4. Boxes of promoters with arrowheads show the direction of the gene sequences. The abbreviations are p for promoter, 3′ for 3′ end of the gene (polyadenylation site), LOX for signal site for excision of DNA sequence by Cre recombinase. Boxes of promoters show the direction of the gene sequences. The constructs in the first and second chromosome I and II are shown separately.

FIG. 6a presents the construct in the first chromosome I is presented to show the principle for placing the genes in detail (upper row) and on a more general level (lower row).

FIG. 6b presents the construct in the second chromosome II in detail and on a more general level.

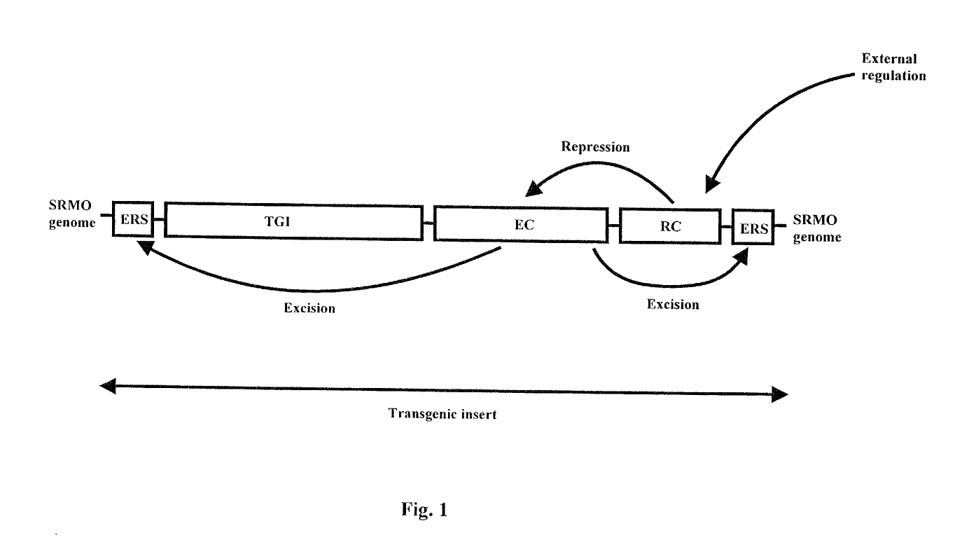

### A DETAILED DESCRIPTION OF THE INVENTION

Abbreviations

cre a gene coding for Cre recombinase

Cre Cre recombinase enzyme

EC excision construct

ERS excision recognition site

GUS β-glucuronidase

hpt hygromycin phosphotransferase

HS heat shock

ICL isocitrate liase

LEA late embryogenesis activated

LOX signal site for excision of DNA sequence by Cre recombinase

MS malate synthase

nos nopaline synthase

nptII neomycin phosphotransferase

ocs octopine synthase

PR-C1 pathogen related C1 gene

RBF recoverable block of function

RC repression construct

RD-RES reverse-delayed repressible excision system

RES repressible excision system

SH-EP sulfhydryl endopeptidase

SRMO sexually reproducing multicellular organism

tet tet operon including tetR gene or tet operator

tetR a gene coding for the tetracycline repressor

TetR tetracycline repressor protein

TGI transgene of interest

Tn10 transposon 10

uidA a gene encoding for β-glucuronidase (GUS)

WT wild type (non-transgenic) line

Definitions

In the present invention most of the terms used have the same meaning as they generally have in the fields of recombinant DNA techniques, molecular biology and in sciences related to plant production. Some terms are, however, used in a somewhat different way and are explained in more detail below.

The term “repressible excision system (RES)” means a molecular control system or molecular control mechanism which comprises at least one excision construct (EC) which responds to externally applicable means for controlling the repression of the expression of the gene encoding the recombinase enzyme. The repressible excision system (RES) performs the control of the segregation and prevents the escape of transgene into related sexually reproducing multicellular organism (SRMO), i.e. plants or non-human animals. The repressible excision system (RES) is introduced into the sexually reproducing multicellular organism (SRMO) together with the transgenes of interest (TGIs) by a process of genetic transformation.

The term “SRMO” means sexually reproducing multicellular organism including both plants and nonhuman animals. Preferred plants are flowering plants and include according to taxonomic classification systems both angiosperms and gymnosperms, especially sexually propagated crop plants, such as cereals. Especially, SRMOs are such sexually reproducing multicellular organisms, which can cross or hybridize with other related cultivated or wild-type species with so called interline hybridization. Preferred animals are for example fish, shrimps, snails, poultry, etc.

The term “transgene(s) or gene(s) of interest (TGIs)” means DNA or nucleotide sequence(s), including RNA sequences, which encode at least one desired gene product, i.e. RNA, a protein or an enzyme or other substances, metabolites, oils, starches, plastic compounds, hormones, alkaloids, vitamins; toxins, vaccines, antibiotics, etc., which are obtainable as end-products by the action of the direct gene products. Said transgenes of interest (TGIs) are introduced into the genome of the SRMO through genetic transformation. The transgenes of interest (TGIs) usually encode products or molecules useful in agriculture, horticulture, forestry and/or industry. Alternatively, the gene products have some other feasible applications. Generally, the nucleotide sequence is not native to the sexually reproducing multicellular organism, the SRMO, but sometimes native nucleotide sequences can be used and inserted e.g. as multiple copies in order to obtain the desired product in higher amounts. In other words, the DNA constructs may consist of one gene or nucleotide sequence or multiples thereof. Alternatively, several different genes of interest can be introduced.

The term “excision recognition sites (ERSs)” means the specific signal sequences, flanking the transgene or gene of interest (TGI) on both sides, which signal sites are recognized by the excising enzyme e.g. recombinase resulting in an excision of the insert.

The term “excision” means generally the process of cutting off a DNA or nucleotide sequence or transgenic insert from the genome of an organism. The excised DNA sequence is flanked by specific recognition sites, such as excision recognition sites (ERSs). In the present invention “excision” is achieved by the excision construct (EC), which is responsive to an outside repression or a molecular control mechanism, either alone or in combination with one or more repression constructs (RCs), e.g. a nucleotide sequence or construct which provides the repression of the expression of recombinase enzyme. In other words it controls the recombinase activity in the host organism at the level of DNA or mRNA. If the externally applicaple means for controlling the recombinase activity are not applied as happens in the environment, the excision mechanism is turned on.

The term “excision construct (EC)” means any DNA construct comprising a nucleotide sequence encoding a recombinase enzyme, capable of excising a nucleotide sequence flanked by appropriate excision recognition sites (ERSs) from the genome. In other words the excision construct (EC) is a combination of nucleotide sequences encoding protein(s)/enzyme(s) capable of excising transgenic inserts flanked by appropriate excision recognizing sites (ERSs). In extreme cases, the excision construct (EC) can be placed in the intron of the TGI. The nucleotide sequence(s) responsible for excising the transgenic insert comprises at least one nucleotide sequence, the action of which leads to expression of an excising enzyme, e.g. the recombinase enzyme able to identify the appropriate signal sequences or excision recognition sites (ERSs) and to excise the transgenic insert flanked by said excision recognition sites (ERSs). The excision construct (EC) is preferably closely linked to the transgene of interest (TGI). At least, it should be placed in the same chromosome providing a so called general or fully repressible excision system. The excision construct (EC) may also be placed between two TGIs. The expression of the gene encoding recombinase in the excision construct (EC) is either constitutive or organ or development specific.

The term “repression construct (RC)” means a DNA construct including one or more nucleotide sequences, the actions of which turns off a gene encoding recombinase as a response to an outside stimulus or turns on an inducible repression mechanism based on outside stimulus or as a result of support or maintenance of the homozygous condition of RES in case of delayed RES. The repression construct (RC) is introduced into the genome of the sexually reproducing multicellular organism, the SRMO, separately or together with the excision construct (EC) and one or more TGIs. The action of the repression construct (RC) is responsive to at least one outside stimulus or is regulated through such hybridization which supports homologous conditions. In other words, it must be externally regulated or externally regulatable. The repression construct (RC) does not act, when not activated by an external manipulation or disappears from genome of the sexually reproducing multicellular organism (SRMO). This occurs under natural conditions when the organism (SRMO) has escaped. Without the externally applicable means the expression of recombinase goes on and the inducible repression is removed, both mechanisms leading to expression of recombinase and excision of the transgenic insert and its disappearance in nature.

The term “excising enzyme” means an enzyme which is encoded by a gene present in the excision construct (EC) and has the capacity of excising transgenic inserts. A representative example of such an enzyme is recombinase. In the present invention the term “recombinase” is used instead of the term “excising enzyme” and covers also other enzymes which may act as excising enzymes.

“Recombinases” are enzymes being capable of excising a DNA sequence flanked by the specific excision recognition sites (ERSs), i.e. specific signal sequences. Several recombinase systems are disclosed in U.S. Pat. No. 4,959,317 and by Sadowski 1993. A preferred recombinase system is the bacteriophage CRE/LOX system, wherein the CRE protein performs a site-specific recombination of DNA in the LOX sites. Alternatively, recombinase may comprise a Flp/frt system and expression silencing may be affected by the expression of antisense RNA of the gene encoding recombinase. In said case the expression of the antisense RNA launches a silencing mechanism on the cre recombinase expression.

The term “externally applicaple means” is used in the present invention for one or more outside stimuli which can be used to regulate or control the repression of the expression of the recombinase enzyme, i.e. preventing the action of the recombinase expressed by the excision construct (EC). The externally applicable means are artificial manipulation steps or treatments, which regulate or control the action of one or more repressing constructs (RCs), which respond to said outside stimuli and are capable of turning off a gene encoding the recombinase enzyme in order to allow the expression of the transgene of interest (TGI) and the production of the transgene product. Alternatively, the external means turn on an inducible repression mechanism. Externally applicable means may include support or maintenance of homozygous conditions in the repressible excision system (RES) in case of delayed RES by introline crossing or reproduction of transgenic lines.

“An externally applicable control” of the repression construct can be provided for example by an outside stimulus of a responsive promoter. The term “repression of the recombinase enzyme action” means an externally regulated molecular mechanism which may include the addition of at least one externally applicable substance, chemical such as a sugar capable of repressing a promoter of the recombinase enzyme. Alternatively, the molecular mechanism may include the application of at least one external chemical or physical stimulus capable of activating the repression mechanism or repression construct which is capable of responding to an outside stimulus and thereby silencing the recombinase RNA expression e.g. by antisense RNA technology. The externally applicable means may for example activate the expression of a protein binding to a specific nucleotide sequence resulting in repression of the recombinase expression. Thus, the repressible excision system (RES) is a system which can be artificially controlled by applying certain specific means which control the genes or constructs which are capable of responding to such outside stimuli and thereby repressing the expression of the recombinase enzyme while producing the desired transgenic product.

The term “escape of a transgene into the environment” means that transgene leaks into nature through hybridization, i.e. crossing of the parental transgenic organism with its wild-type or cultured non-transgenic relatives. In other words, in the present invention the release of the transgene is prohibited. This is achieved by allowing the excision construct (EC) to function under unrepressed, i.e. normal, natural, unmanipulated, treatment-free or intervention-free conditions or uncontrolled environment. Said natural, unmanipulated conditions remove or leave inactive the repressing functions and the excising recombinase enzyme is free to act. Accordingly, the transgenic insert in the sexually reproducing multicellular organism (SRMO), i.e. the plant or animal is lost or disintegrates under natural conditions when the repression achieved by an external artificial means does not work. If an external artificial manipulation is applied the repression system is turned on or is supported in such a way that a functional recombinase is not produced and the transgenic plant carries out the desired functions of the transgene insert.

The term “natural conditions” means the growth conditions, which are the usual ambient parameters of temperature, humidity, irradiation, chemical background of soils in the natural environment of corresponding wild-type or the cultivated non-transgenic sexually reproducing multicellular organism (SRMO), including plant or animal growth in agriculture, horticulture, forestry or in the nature.

The term “repression of recombinase expression” means lack of recombinase action as a result of the action of the repression mechanism. The action of the promoter or expression of mRNA of the gene encoding recombinase can be repressed by the action of a repression construct (RC).

The term “general RES” is a synonym for “full RES” which is the prototype embodiment of the RES-systems. It means that the repression construct (RC) is placed in the same chromosome with the excision construct (EC) and the transgene of interest (TGI) resulting in a so called general repression in the parent generation including expression of desired transgenes under control condition and excision if the transgene escapes.

The term “delayed RES” means a repressible excision system (RES), wherein the repression construct (RC) is situated in a different non-allelic chromosome in the same organism, i.e. apart from the excision construct (EC) and the gene(s) of interest (TGI). This means that the functions of the excision construct (EC) does not start to work before the second heterozygous hybrid generation, when the excision and repressing constructs (EC and RC) segregate to different generative cells.

The term “reversed delayed RES (RD-RES)” means a reversed delayed repressible excision system (RES) which is obtained when the first excision and repression constructs (EC₁ and RC₁) are located in different non-allelic chromosomes and the second repression construct (RC₂) is linked to the first excision construct (EC₁), linked to the first repression construct (RC₁) and the second excision construct (EC₂) linked to the first repression construct (RC₁) is repressed with a second repression construct (RC₂) linked to the transgene of interest (TGI) and the first excision construct (EC₁).

The term “double RES” means that the gene(s) of interest (TGIs) is (are) situated between two preferably different excision constructs (ECs), which are repressed by the same or different repression mechanisms resulting in a double repressible excision system (double RES). Also complex systems comprising one or more repressible excision systems (RESs) and/or one or more recoverable block of function (RBF) systems in different combinations.

The General Description of the Invention

The present invention is related to a method for molecular control of transgene segregation in the progeny and its spread in populations of related wild sexually reproductive multicellular organisms (SRMOs). The invention describes a method for preventing transgene leakage through hybridization. The control of segregation of the transgene of interest (TGI) is performed by a molecular repressible excision system (RES). The repressible excision system (RES) is a method consisting conceptually of two components: recombinase (DNA excision) construct(s) and the repression system. The gene encoding recombinase is present in a so called excision construct (EC) and is a DNA sequence introduced into the genome of the sexually reproducing multicellular organism (SRMO), i.e. a plant or a non-human animal and is linked to the transgene(s) of interest (TGI). The action of the recombinase leads to excision of the transgenic insert placed between excision recognition sites (ERSs), i.e. the signal sequences recognising a recombinase. Repression of the recombinase expression ensures that the transgenic insert remains unexcised in the genome of sexually reproducing multicellular organism (SRMO) genome as long as the constructs are under control and used for production of the desired gene products. The action of the repression construct (RC) must respond to an external, artificial manipulation step. The repression construct (RC) does not act or lacks its functionality during the life cycle or hybridization under natural, uncontrolled conditions leading to automatic excision and disintegration of the transgenic insert if the transgenic organism escapes.

The present invention is based on several principles. The most preferred embodiment of the present invention is the excision of a DNA sequence from an eucaryotic genome, preferably based on the cre recombinase technology, including interaction of excision and repression constructs (EC and RC) in a similar way as described in the recoverable blocking of functions (RBF) systems described in U.S. Ser. No. 09/617,543, and a suppression of the gene expression, preferably based on the Tn10 tet operator-repressor technology or an antisense mRNA technology.

The present method differs from that described in U.S. Ser. No. 09/617,543 in that instead of the control of the transgene escape through the blocking of a vital function of the host, which is a sexually reproducing multicellular organism (SRMO), the invention implies to control the escape through regulated excision of the transgenic insert from the host SRMO genome, whereas non-transgenic functions of sexually reproducing multicellular organism remain unchanged. Instead of a blocking construct at least one excision construct (EC) is used and instead of using a recovering construct to recover the blocked function a repressing construct (RC) is used. The transgenic function is supported in the sexually reproducing multicellular organism (SRMO) by regulated repression of the excision function. When the repression of the excision is removed, which happens automatically in nature, the recombinase enzyme excises a transgenic insert between the excision recognition sites and the escape of the transgene is prevented.

Thus, the present invention is related to a method for preventing the escape of a transgene into the environment by the excision of transgenic inserts from genomes of sexually reproducing multicellular organisms (SRMOs), especially plants. The excision process is controlled by a DNA construct, a so called excision construct (EC) introduced together with the transgene of interest (TGI) into the genome of a sexually reproducing multicellular organism (SRMO). The excision is performed by a recombinase enzyme. The expression of the gene encoding recombinase can be repressed by a repression mechanism, which is dependent of a construct encoding an element capable of repressing the expression. The expressed recombinase capable of excising the transgenic insert is linked to one or more transgene(s) of interest (TGI). The whole construct containing at least the transgene(s) of interest (TGIs) and the gene encoding recombinase and its function facilitating regulatory or regulating sequences is flanked on both sides by specific excision recognition sites (ERSs).

The recombinase action is repressible by a repressor molecule e.g. an antisense mRNA or a DNA binding protein, which is encoded by a repression construct (RC). The expression of the repression construct (RC) is regulated externally either through promoter stimulation or control of the segregation of the respective constructs. Whenever the transgenic organism is left to grow without human or artificial control, the repression of the recombinase expression will be lost and the transgenic insert will be excised from the genome as a result of the recombinase action. Thus, the transgenic insert will be removed from the genome of the organism.

The present invention is related to molecular techniques controlling transgene escape in natural populations. The method implies genetic transformation of a sexually reproducing multicellular organisms (SRMO). Transgene(s) of interest (TGIs) introduced in the genome of the SRMO is (are) linked to the recombinase gene. The complete transgenic insert, with the exception of left and right borders of T-DNA in case of Agrobacterium transformation, is flanked on both sides by specific excision signal sequences or excision recognition sites (ERSs). The expression of the gene encoding the recombinase enzyme is repressible. The repression can be externally regulated. The control or regulation is organized so that the repression is removed under natural conditions, which lack any means for externally applicable regulation. Whenever the repression is removed or lost, the recombinase will excise the transgenic insert from the genome of the SRMO.

The gene encoding the recombinase should preferably be repressed by the action of a repression construct (RC). Such repression constructs (RC) can be constructed based on any of the promoter repression systems (Lanzer and Bujard 1988), i.e. lac repressor (Figge et al. 1988) or the repressor system activated by a chemical ligand (U.S. Pat. No. 5,880,333). The preferred system is the Tn10 tet repressor system (Gatz and Quail, 1988; Gatz, et al. 1991, 1992; Roder et al., 1994). For example, in the case of the Tn10 tet system repression construct (RC) encodes the TetR repressor protein that binds to the tet operator introduced in the promoter regulating recombinase expression (Example 1). The repression of recombinase expression can be performed by antisense RNA technology (Example 2).

The location or position of the transgene of interest (TGI), recombinase and repression construct (RC) depends on the model used. The structure and the position of the repressible excision systems (RES) are as described for the recoverable block of function (RBF) constructs controlling the transgene escape described in U.S. Ser. No. 09/617,543 The preferred positions are: general repressible excision system (RES) (Example 1) and delayed repressible excision system (RES) (Example 2).

A general repressible excision system (RES) implies that all components, including transgene of interest (TGI), excision and repression constructs (EC and RC) are placed in the same transgenic insert, which is flanked on both sides by specific recombinase signal sequences, i.e. the excision recognition sites (ERSs). Regulation of the expression of the recombinase and the genes responsible for repression can be organized in different ways. Recombinase can be expressed in a constitutive or organ- or development-specific manner, i.e spatiotemporally. The repression construct (RC) is controlled for example by a promoter which is capable of responding to an external stimulus in the case of general repressed excision system (RES). Constitutive expression of the recombinase requires continuous expression of the repression construct (RC) in order to keep the transgenic insert intact and capable of expressing the desired gene product. Organ or development specific expression of recombinase requires only temporal induction of the repressor mechanism, which should coincide with the expression of the promoter controlling the recombinase expression. The particular model of the general repressible excision system is described in Example 1.

The delayed repressible excision system (RES) implies that the transgene of interest (TGI) is linked to the gene encoding recombinase. The entire construct is flanked on both sides by specific excision recognition sites (ERSs). In one specific embodiment of the invention the repression construct (RC) is placed on a different non-allelic chromosome. Both recombinase and repression construct (RC) can be expressed under constitutive or organ-development-specific promoters. The most important trait of the system is that the repression should be strong enough to prevent the recombinase action. The particular model of the delayed repressible excision system (RES) is described in more detail in the Example 2.

The delayed repressible excision starts from the second hybrid progeny, when the recombinase and repression construct (RC) begin to segregate into different individuals. The segregation and interaction of the constructs is analogous to that described in U.S. Ser. No. 09/617,543. The control of the transgene escape can be organized in general, delayed, reversed, reversed-delayed, double, triple, or multiple excision systems analogous to the recoverable block of function (RBF) systems described in U.S. Ser. No. 09/617,543. The general principles for some preferred repressible excision systems (RES) are described below.

The Cre/lox Recombinase-excision System from Bacteriophage P1

The cre recombinase gene is originally responsible for a site specific insertion of the P1 phage (Stenberg et al. 1986) into a loxB site of the chromosomal DNA of Escherichia coli. The P1 phage carries a specific site for recombining loxP, i.e. the locus of crossing-over. The recombination results in integration of the P1 phage into the genome of E. coli with formation of flanking loxR and loxL sites (Hoess et al. 1982). The loxP site has been shown to be the most active for the recombination process (Hoess and Abremski, 1984). It has been shown that the DNA sequence flanked by two directly orientated loxP sites can be effectively excised by a Cre recombinase (Abremski et al. 1983) which has also been shown to be highly effective in plants (Dale and Ow 1990; Russel et al. 1992). The system was used for excising the selectable marker genes from the plant genome (Russel et al. 1992; Gleave et al. 1999). The cre recombinase gene expression and the modified loxP sites were used for constructing the so called “terminator technology” (U.S. Pat. No. 5,723,765).

The recombination systems R/rs from Zygosaccharomyces rouxii and Flp/frt from Saccharomyces cerevisiae, require only the gene encoding recombinase and the target sequences for recombination and have been shown to function in plants (Onouchi et al. 1991; Pan et al. 1993; Kilby et al. 1995). Other recombinase systems include the Tn21 resolvases (Hall and Halford, 1993), the SSV1 encoded integrase (Muskhekishvili et al. 1993) and the Ac/Ds transposon system in maize (Shen and Hohn, 1992).

Preferred Repression Construct (RC) Systems

DNA binding proteins can be used for blocking the promoter function of the recombinase or for blocking the recombinase target (recognition) sites, i. e. 35Sp of loxP. The transposon Tn10 has been shown to carry the tet operon including the tetR repressor and the tet operator target sequences. The tetR gene encodes a TetR repressing protein and specifically binding target tet operator sequences in the promoter of tet (tetracycline) resistance gene (Hillen et al. 1984). The highly expressed and purified TetR repressor protein has been shown to bind the tet operator sequences (Oehmichen et al. 1984). The tet repressor-operator system has been shown to function in modified CaMV 35S promoter in plants (Gatz and Quail 1988; Gatz et al. 1991). The precise effective positions of the tet operator in relation to as-1 and TATA boxes were determined in CaMV 35S promoter (Fronberg et al. 1991). The most efficient 500-fold repression of the CaMV 35S promoter was achieved by introducing three tet operator sequences into the promoter region (Gatz et al. 1992). The tet system would be the preferred promoter repression mechanism, if high expression of the TetR protein would not cause toxicity symptoms in some plants, such as Arabidopsis and tomato (Corlett et al. 1999). Other repressor systems based on a protein—DNA binding process have been disclosed by (Lanzer and Bujard, 1988), and include the lac repressor (Figge et al. 1988) or repressor system activated by a chemical ligand in plants (U.S. Pat. No. 5,880,333).

The silencing of gene expression by the production of antisense RNA has been described for different genes in different plants. Said mechanism can be used for permanent repression of recombinase expression. If the recombinase gene would be controlled by an organ or development specific promoter, the silencing could be supported by a permanent expression of a part of the recombinase mRNA (at least 200 nucleotides) in sense and antisense orientation. The antisense RNA expression is preferably used for the delayed RES construction.

Several promoters useful for the regulation of the expression of the excision (EC) and/or repression constructs (RC) are presented below. Promoters for constitutive expression are for example the CaMV 35S promoter (Condit et al., 1983; Zaitlin et al., 1985; Williamson et al., 1989) the NOS-promoter (Depicker et al., 1982) or the OCS-promoter (De Greve et al., 1985).

Preferred Organ and Development Specific Promoters

Several promoters show high seed germination specificity in the expression. Unfortunately, each promoter has also some additional non-specific expression at other stages of development. SH-EP or EP-C1 cysteine endopeptidases are known to specifically degrade storage proteins. They are proteins, with a high expression in germinating seeds. Additional expression can be observed in senescent tissues: cotyledons, pods and even in stems. These genes have large promoter regions of about 1200-1700 bp. EP-C1 cysteine endopeptidase has been cloned from Phaseolus vulgaris (Ogushi et al., 1992) and expressed in a deletion analysis series in transgenic tobacco seedlings (Yamauchi et al., 1996).

SH-EP promoter of sulfhydryl endopeptidase (1676 bp) has been cloned from Vigna mungo (Akasofu et al., 1990) and investigated as SH-EP-cysteine endopeptidase (Yamauchi et al., 1996). It is mostly expressing in germinating seeds and exhibits non-specific activity during embryo development.

Late embryogenesis activated (LEA) promoters express during late embryo development stages (Hughes and Galau, 1989; Galau, et al., 1992; Devic, et al., 1996). LEA promoters are not “leaky” and are highly specific to late embryogenesis.

Malate synthase (MS) and isocitrate liase (ICL) promoters which are suppressed by sucrose, activated by GA3 are active during germination and in seedlings. Additional expression occurs in senescing organs and in germinating pollen. Promoters from cucumber (Reynolds and Smith, 1995), oilseed rape (Zhang et al., 1994) and tomato (Janssen, 1995) have been thoroughly investigated in spatiotemporal manner in transgenic tobacco and oilseed rape. Malate synthetase (MS) has a wider expression spectrum and is less ideal for germination specific expression (Sarah et al., 1996).

A 17 bp fragment responsive to gibberellin from a promoter of catrepsin B-like protein of wheat (Cejudo et al., 1992) has been reported. The AMY (high P1)-alpha-amylase is active in the endosperm during germination. All the known amylase promoters are also active in different organs of Phaseolus vulgaris and Vigna mungo plants (Minamikava et al., 1992). There is a known GA3 induced P1 amylase from barley (Rahmatullah et al., 1989). Beta-1,3 glucanase is active in the endosperm during germination. This is an antifungal protein induced by GA4. It is active also in leaves and other organs, and is wound inducible (Vgeli-Lange et al., 1994). There is a number of other candidate promoters for temporal expression and the number increases yearly.

Promoters Responsive to Outside Stimulus

The outside stimulus can be chemical or physical. The chemical stimulus can be any molecule capable of regulating the activity of a particular promoter. The physical stimulus can be temperature, osmosis, light, gravitation, etc.

As examples of two types of promoters responding to outside stimulus, the salicylate and heat shock (HS) inducible promoters, can be mentioned. The salicylate inducible promoters are related to virus or other pathogen infections and are specially involved in stress responses. Promoter of the pathogenesis-related PA1 gene has been studied in the 5′ deletion experiments performed in transgenic tobacco (Ohshima et al., 1990). Several regulatory elements have been found in the 902 bp PR-1a promoter in another deletion experiment (Van de Rhee, et al., 1990; Payne, et al., 1988; Pfitzner et al., 1988). The activity of the promoters rose after 24-48 hours of salicylate induction and was close to the level induced by TMV infection. The heat shock (HS) promoters have been investigated in depth in different plants. Their activity has been shown to be several times higher than that of the CaMV 35S promoter. The induction of the heat shock (HS) promoters usually occurs when the ambient temperature rises to 35-45° C. (Czarnecka, et al., 1989). The drawbacks of the heat shock (HS) promoters are their expression in seedlings and their activation also by other physical stimulus than heat.

Chemically controlled expression of promoters has been disclosed in U.S. Pat. No. 5,880,333. The Tn10 tet repressor system, which was developed by Gatz and Quail (1988) should preferably be activated by the chemical agent, tetracycline. In the present invention the induction mechanism, however, opposite to the standard induction mechanism requires two genes. The first gene is the recombinase gene driven by the promoter containing the tet operator. The second gene is positioned under a constitutive or organ/development responsive promoter gene encoding the repressor protein, which binds the tet operator DNA signal sequence. The system stays under repression until externally applied tetracycline activates the recombinase gene.

The externally regulated repressible excision system (RES) can be used for excising the transgene in a desired stage of cultivation by inactivating the repression function. For example, the transgenic insert can be excised by adding tetracycline, which removes the repression caused by the TetR repression construct (RC) described in Example 1. The removed transgene can perform a function undesired in further cultivation or processing of the sexually reproducible multicellular organism (SRMO).

The invention is illustrated by the following concrete examples of the constructs and how they work.

### EXAMPLE 1

General Repressible Excision System (RES)—One Transgenic Insert in Tobacco Plants

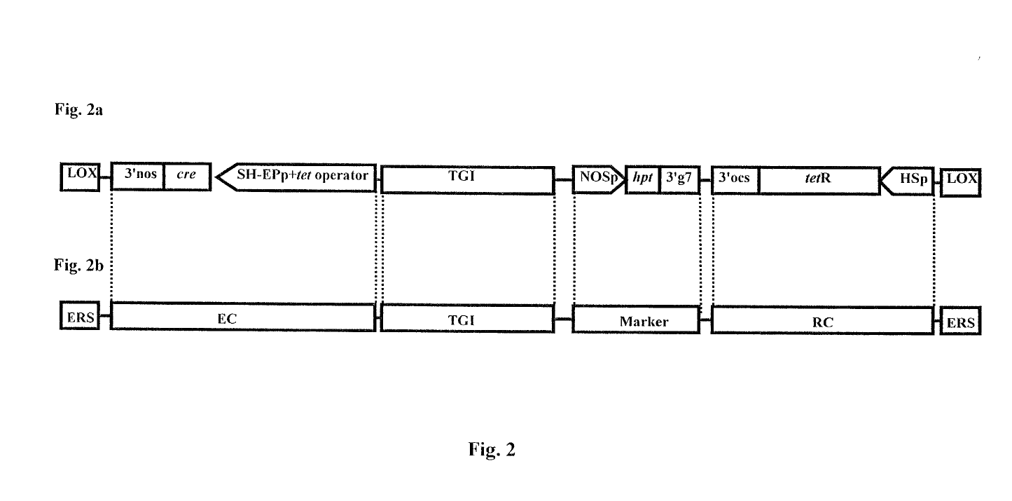

The transgenic insert comprises the GUS (uidA) gene as a representative of a transgene of interest (TGI), the cre recombinase gene and the Tn10 tet operator-repressor system. The insert is flanked by loxP excision recognition sites (ERSs). The GUS gene is placed under the control of CaMV 35S promoter. The chimeric cysteine-endopeptidase promoter (SH-EPp) from Vigna mungo (Akasofu, et al., 1990), with inserted tet operator sequences, regulates the cre recombinase expression. The Tn10 tet repressor gene is regulated by the heat shock promoter (HSp) from soybean (Czarnecka, et al. 1989). The model is schematically shown in FIG. 2. Additionally, the loxP sites are flanked on both sides by tet operator sequences. Alternatively, other promoters can be used. For example, cre recombinase can be regulated by modified late embryogenesis activated (LEA) or malate synthase (MS) promoter. The Tn10 tet repressor gene can be regulated by pathogen related C1 (PR-C1) promoter induced by salicylate.

The tobacco plants carrying a transgenic insert loose the insert when they are grown under uncontrolled or natural conditions. Thus, subsequent generations of the tobacco plant do not contain a transgene. To keep the transgenic insert in the genome, the transgenic tobacco plant should be treated with a temperature higher than the ambient temperature (e.g. +40° C.) for one hour every second day during seedpod maturation. After the heat shock (HS) treatment the plants keep the transgenic insert in the genome. Under natural conditions cre recombinase is expressed by chimeric SH-EP promoter in the embryos during seedpod maturation and the transgenic insert is excised from the genome. The heat shock (HS) treatment activates TetR repressor expression during seedpod maturation. The repressor protein binds tet operator in the chimeric SH-EP promoter and represses the cre recombinase expression. Therefore, after the heat shock (HS) treatment, the seeds contain the non-excised transgenic insert in the genome. Under natural or uncontrolled conditions, where the heat shock (HS) treatment would not be applied, the plants would loose the transgenic insert placed between loxP sites in the result of the action of the cre recombinase.

### EXAMPLE 2

Delayed Repressible Excision System (RES) in Tobacco and Turnip Rape

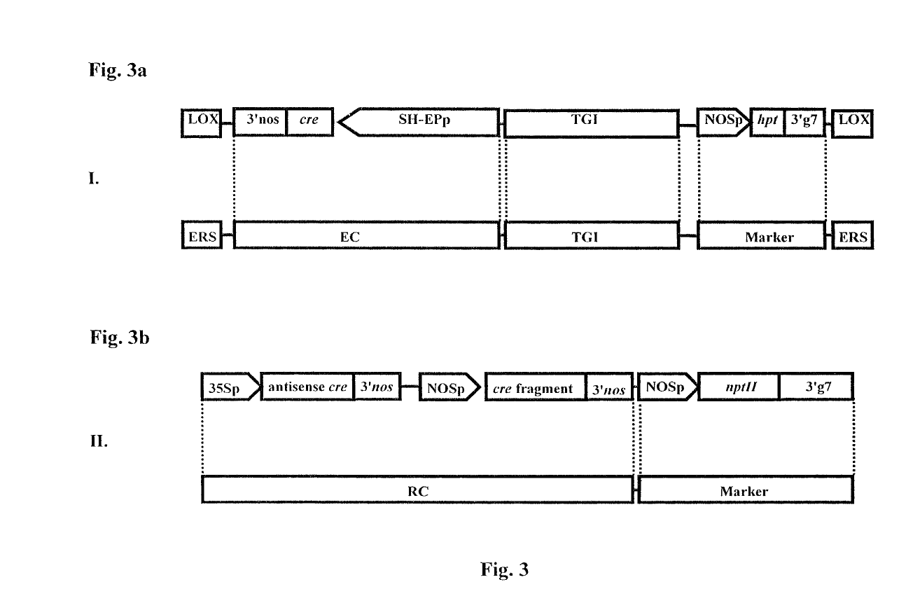

The first transgenic insert contains the GUS (uidA) gene as a representative of the transgene of interest (TGI) linked to the cre recombinase gene. The insert is flanked by the specific lox excision recognition sites (ERSs). The second transgenic insert is located on a different non-allelic chromosome and contains a part of a sense cre RNA and an antisense cre mRNA. The GUS gene is placed under the control of the CaMV 35S promoter. The cre gene is placed under the control of the NOS promoter (NOSp). The antisense cre gene is regulated by the CaMV 35S promoter. A 600 bp long fragment of cre gene is regulated by NOSp. Both constructs are in homozygous condition. The promoter of the cre recombinase is continuously repressed by an antisense RNA silencing mechanism. The DNA constructs are schematically shown in FIG. 3.

Introline crossing keeps both of the transgenic inserts in homozygous condition, and the recombinase gene remains under continuous repression. As a result of the outside hybridization or out-crossing with plants without the recombinase-repressor system, plants of the first hybrid progeny have the transgenic inserts in heterozygous condition. The out-crossed hybrid population begins to loose the transgenic insert containing the transgene of interest (TGI). From the second hybrid generation, only one-fourth of the plants contain the transgenic insert with the transgene of interest (TGI). Half of the transgenic inserts are lost among the transgenic progeny as a result of the excision. The delayed excision can be used in different molecular systems to control transgene escape in a similar way as that described in U.S. Ser. No. 09/617,543.

### EXAMPLE 3

Combined RBF and RES Systems in Triple RBF/RES

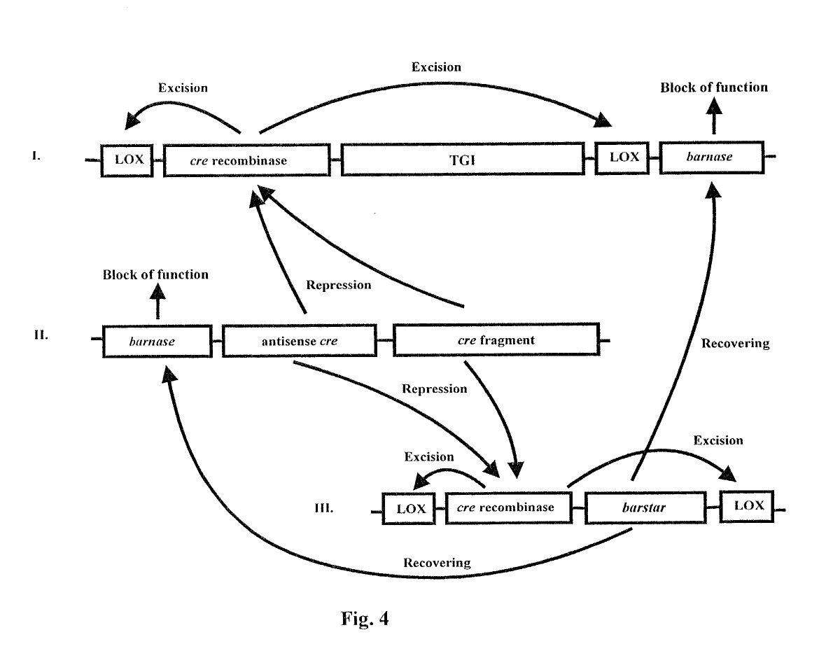

The schematic structures of the constructs used in a combined recoverable block of function (RBF)/repressible excision system (RES) are presented in FIG. 4. Three transgenic inserts are introduced into different non-allelic chromosomes and are in homozygous conditions. The first insert contains cre recombinase gene under the control of nos promoter and a barstar gene under the control of the CaMV 35S promoter. The insert is flanked by LoxP sites in the same orientation. The second insert contains the barnase gene under the control of the chimeric cysteine-endopeptidase (SH-EP) promoter as well as the antisense cre and 600 bp fragment of the sense cre recombinase, both under the control of the CaMV 35S promoters. The third insert contains transgene(s) of interest (TGIs) flanked by cre recombinase on one side and barnase on the other side of the insert. The LoxP sites are placed in the insert as shown in FIG. 4. The interaction of the constructs is shown by arrows.

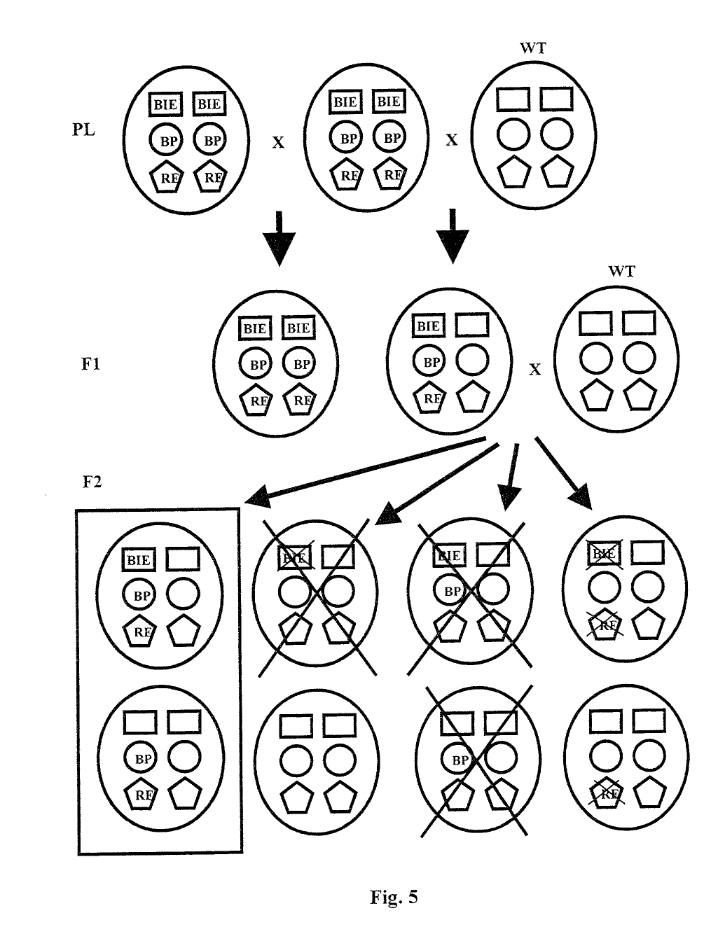

The segregation of the constructs during introline and outside crossing is the same as in triple recoverable block of function (RBF) described in U.S. Ser. No. 09/617,543 and is shown in FIG. 5. Slashed constructs are excised as a result of the free action of the excision construct (EC) in the repressible excision system (RES). Slashed genomes are suicidal or sterile due to the free action of the blocking construct (BC) in the recoverable block of function (RBF) system.

### EXAMPLE 4

Combined Delayed/General Repressible Excision System (RES)

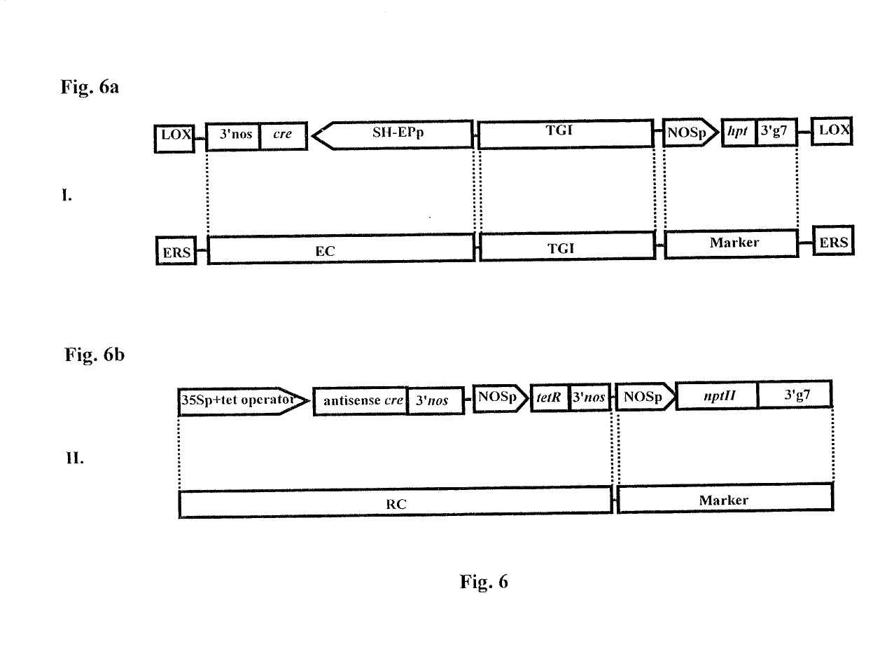

The first transgenic insert contains the GUS (uidA) gene under the CaMV 35S promoter as a representative of the transgene of interest (TGI) linked to the cre recombinase gene. The insertion is flanked by the specific lox excision recognition sites (ERSs). The second transgene insertion located in a different non-allelic chromosome contains the Tn10 tet repressor gene regulated by NOS promoter and antisense cre mRNA expressed under chimaeric CaMV 35S promoter containing three tet operators. The cre gene is placed under the control of the SH-EP promoter. Both constructs are in homozygous condition. The promoter of the antisense cre recombinase is repressed by continuously expressed TetR repressor protein. Addition of 0.1 mg/ml tetracycline changes the conformation of TetR repressor protein, releases chimaeric CaMV 35S promoter and activates the repression of the cre recombinase through silencing mechanism provoked by expression of antisense and sense mRNA of cre recombinase. Schematically the DNA constructs are shown in FIG. 6.

Introline crossing keeps both of the transgene inserts in homozygous conditions, although, to produce alive seeds, the recombinase gene requires activation of its repression by application of external stimuli—tetracycline at the stage of embryo development and germination. As a result of outside hybridization (out breeding) with plants without the recombinase-repressor system, plants of the first hybrid progeny have the transgene insertion in heterozygous condition. The out-cross hybrid population begins to loose the transgenic insertion containing the gene of interest (TGI) in the result of two factors: loss of repression construct (RC) and absence of external stimuli activating the repression construct (RC) (tetracycline treatment). If even TetR repression protein lack to function and repression of excision construct (EC) would be continuous, only one-fourth of the plants will contain the transgenic insertion with the gene of interest (TGI) from the second hybrid generation.

References

Abremski, et al., 1983, Cell 32: 1301-1311

Akasofu, et al., 1990, Nucl. Acids Res. 18: 1892

Cejudo, et al., 1992, Plant Mol. Biol. 20: 849-856

Condit, et al., 1983, J. Mol. Appl. Gen. (USA) 2: 301-314

Corlett, et al., 1999, Plant Cell Environ. 19: 447-454

Czarnecka, et al., 1989, Mol. Cell Biol. 9: 3457

Dale and Ow, 1990, Gene 91: 79-85

De Greve, et al., 1985, J. Mol. Appl. Gen. (USA) 1: 499-511

Depicker, et al., 1982, J. Mol. Appl. Gen. (USA) 1: 561-573

Devic, et al., 1996, Plant J. 9: 205-2015

Figge, et al., 1988, Cell 52: 713-722

Fronberg, et al., 1991, Proc. Natl. Acad. Sci. USA 88: 10470-10474

Gatz and Quail, 1988, Proc. Natl. Acad. Sci. USA 85: 1394-1397

Galau, et al., 1992, Plant Physiol. 99: 783-788

Gatz, et al., 1991, Mol. Gen. Genet. 227: 229-237

Gatz, et al., 1992, Plant J. 2: 397-404

Gleave, et al., 1999, Plant Mol. Biol. 40: 223-235

Hillen, et al., 1984, J. Mol. Biol. 172: 185-201

Hoess, et al., 1982, Prc. Natl. Acad. Sci. USA 79: 3398-3402

Hoess and Abremski, 1984, Proc. Natl. Acad. Sci. USA 81: 1026-1029

Hudspeth, et al., 1996, Plant Mol. Biol. 31: 701-705

Huges and Gallau, 1989, Genes Dev. 3: 358-369

Janssen, 1995, Plant Physiol. 108: 1339

Kilby, et al., 1995, Plant J. 8: 637-652

Lanzer and Bujard, 1988, Proc. Natl. Acad. Sci. USA 85: 8973-8977

Minamikava, et al., 1992, Plant Cell Physiol. 33: 253-258

Muskhelishvili, et al., 1993, Mol. Gen. Genet. 237: 334-342

Oehmichen, et al., 1984, EMBO J. 3 539-543

Ogushi, et al., 1992, Plant Mol. Biol. 19: 705-706

Ohshima, et al., 1990, Plant Cell 2: 95-106

Onouchi, et al., 1991, Nucl. Acids Res. 19: 6373-6378

Payne, et al., 1988, Plant Mol. Biol. 11: 89-94

Pfitzner, et al., 1988, Mol. Gen. Genet. 211: 290-295

Rahmatullah, et al., 1989, Plant Mol. Biol. 12: 119-121

Reynolds and Smith, 1995, Plant Mol. Biol. 27: 487-497

Roder, et al., 1994, Mol. Gen. Genet. 243: 32-38

Russell, et al., 1992, Mol. Gen. Genet. 234: 49-59

Sadowski 1993, The FASEB J.7:760-767

Sarah, et al., 1996, Mol. Gen. Genet. 250: 153-161

Shen and Hohn, 1992, Plant J. 2: 35-42

Stenberg, et al. 1986, J. Mol. Biol. 187: 197-212

Van de Rhee, et al., 1990, Plant Mol. Biol. 21:451-461

Vgeli-Lange, et al., 1994, Plant J. 5: 273-278

Williamson, et al., 1989, Plant Physiol. 90: 1570-1576

Zaitlin, et al., 1985, Biotechnology in plant science: relevance to agriculture in the eighties. Orlando, Fla. (USA). Academic press, p. 227-235

Zhang, et al., 1994, Plant Physiol. 104: 875-864

Yamauchi, et al., 1996, Plant Mol. Biol. 30: 321-329

We claim.

1. A method for controlling transgene segregation in a sexually reproducing multicellular organism (SRMO) and for preventing the escape of said transgene into the environment by a molecular mechanism including a repressible excision system (RES), said method comprising the steps of
(a) providing, functionally integrated into the genome of a sexually reproducing multicellular organism (SRMO), DNA constructs which have the capacity of responding to externally applicable means and which comprise
(i) one or more transgenes of interest (TGIs) encoding desired gene products;
(ii) said transgenes of interest (TGIs) being closely linked to an excision construct (EC) comprising at least one gene encoding a recombinase enzyme, which is operably linked to one or more regulating sequences or repression constructs (RCs), which comprise regulating sequences which are capable of repressing the expression of the gene encoding the recombinase enzyme and responding to at least one externally applicable means alone or under the control of one or more additional repression constructs (RCs);
(iv) said excision constructs (ECs), transgenes of interest (TGIs), regulating sequences and/or optional repression constructs (RCs) forming a transgenic insert flanked by excision recognition sites (ERSs);
(b) applying at least one externally applicable means, which maintain/support the repression of the expression of the recombinase enzyme through the regulating sequences or constructs operably linked to the gene encoding recombinase with or without the optional interaction of one or more additional repression constructs (RCs) placed in the same or different non-allelic chromosomes. and which means simultaneously allow the expression of the transgene of interest (TGI); and
(c) providing automatic excision of the transgenic insert flanked by the excision recognition sites (ERSs) when the repression of the recombinase expression ends at the withdrawal of the externally applicable means.

2. The method according to claim 1, wherein the expression of the gene encoding the recombinase enzyme is constitutive or an organ or development specific.

3. The method according to claim 2, wherein the constitutive or organ or development specific expression of the gene encoding the recombinase enzyme is controlled by a regulating sequence or a promoter.

4. The method according to claim 3, wherein the expression of the gene encoding the recombinase enzyme is controlled by a CaMV 35S promoter or a nopaline synthase (NOS) promoter.

5. The method according to claim 3, wherein the expression of the gene encoding the recombinase enzyme is controlled by an organ or development specific cysteine endopeptidase (SH-EP) promoter.

6. The method according to claim 1, wherein the expression of the repression construct (RC) is constitutive or inducible.

7. The method according to claim 1, wherein the constitutive or inducible expression of the repression construct is controlled by a regulating sequence or a promoter.

8. The method according to claim 7, wherein the expression of the repression construct (RC) is controlled by the CaMV 35S or nopaline synthase (NOS) promoter.

9. The method according to claim 7, wherein the inducible expression of the repression construct (RC) is controlled by an inducible heat shock (HS) promoter.

10. The method according to the claim 9, wherein the promoter is the heat shock (HS) promoter induced by rising the temperature above an ambient temperature.

11. The method according to claim 2, wherein the constitutive expression of the gene encoding the recombinase enzyme is continuously repressed by a constitutive expression of the repression construct (RC).

12. The method according to claim 2, wherein the expression of the gene encoding the recombinase enzyme is repressed by expression of the repression construct (RC) which has the capacity to respond to externally applicable means.

13. The method according to the claim 1, wherein repression of the expression of the gene encoding the recombinase enzyme is obtainable by methods selected from a group consisting of
(a) blocking the action of the promoter of the gene encoding recombinase enzyme present in an excision construct (EC) and
(b) allowing a nucleotide sequence present in a repression construct (RC) to express an antisense RNA of the gene encoding said recombinase enzyme present in the excision construct (EC).

14. The method according to the claim 13, wherein repression of the expression of the gene encoding the recombinase enzyme comprises the blocking of the action of the promoter of the gene encoding the recombinase enzyme present in the excision construct (EC).

15. The method according to the claim 13, wherein the blocking of the promoter action is obtained by introducing one or more operator sequences into the promoter of the gene encoding the recombinase enzyme.

16. The method according to the claim 13, wherein repression of the recombinase enzyme expression is achieved by allowing a gene present in the repression construct (RC) to express a DNA binding protein, which is capable of preventing the gene encoding recombinase from functioning.

17. The method according to the claim 13, wherein the gene in the repression construct (RC) expresses a repressor protein having the ability to bind to the operator sequence(s) introduced into the promoter sequence of the gene encoding said recombinase enzyme and thereby to repress the expression of the recombinase enzyme or/and in the vicinity of the excision recognition site (ERS).

18. The method according to the claim 13 allowing a gene present in the repression construct (RC) to express a protein which is capable of binding to the promoter of said recombinase enzyme and thereby preventing it from functioning.

19. The method according to the claim 13, wherein the gene encoding the recombinase enzyme is a cre recombinase and the repression construct (RC) comprises a Tn10 tet repressor—operator system.

20. The method according to the claim 13, wherein repression of the recombinase enzyme expression is achieved by allowing a nucleotide sequence present in the repression construct (RC) to express an antisense RNA of the gene encoding said recombinase enzyme.

21. The method according to the claim 19, wherein the nucleotide sequence present in the repression construct (RC) and expressing an antisense mRNA of the recombinase enzyme is antisense cre recombinase RNA, which launches a silencing mechanism on the excision construct which is responsible for the cre recombinase expression.

22. The method according to the claim 1, wherein the repression of recombinase enzyme action comprises externally applicable means capable of controlling a molecular mechanism responsible for the expression and repression of the gene encoding the recombinase enzyme.

23. The method according to claim 22, wherein externally applicable means for repressing the recombinase enzymes is selected form a group consisting of
(a) adding at least one externally applicable substance;
(b) applying at least one external chemical or physical stimulus capable of activating the repression mechanism;
(c) repressing the promoter of the recombinase enzyme;
(d) silencing the expression of the recombinase RNA by antisense RNA technology;
(e) expressing a protein binding to a specific regulating nucleotide sequence resulting in repression of the recombinase enzyme expression; and
(f) supporting/maintaining the excision construct (EC) and repression construct (RC) in homozygous conditions through an introline crossing when excision construct (EC) and repression construct (RC) are located in different non-allelic chromosomes.

24. The method according to claim 23, wherein externally applicable means comprises the addition of at least one externally applicable substance.

25. The method according to claim 23, wherein externally applicable means comprises the application of at least one external chemical or physical stimulus capable of activating the repression mechanism.

26. The method according to claim 23, wherein externally applicable means comprises the repression of the promoter of the recombinase enzyme;

27. The method according to claim 23, wherein externally applicable means comprises silencing the recombinase RNA expression by antisense RNA technology.

28. The method according to claim 23, wherein externally applicable means comprises the expression a protein binding to the specific nucleotide sequence resulting in repression of the recombinase enzyme expression.

29. The method according to claim 23, wherein externally applicable means comprise the introline crossing to maintain homozygous condition of excision construct (EC) and repression construct (RC), when the excision construct (EC) and repression construct (RC) are located in different non-allelic chromosomes.

30. The method according to claim 23, wherein the externally applicable means comprises the addition an effective amount of tetracycline.

31. The method according to claim 1, wherein a general repressible excision system is provided by placing the excision and repression constructs (EC and RC) in the same chromosome.

32. The method according to claim 1, wherein a delayed repressible excision system (RES) is provided by placing the excision and repression constructs (EC and RC) in different non-allelic chromosomes.

33. The method according to claim 1, wherein a reversed delayed repressible excision system (RD-RES) is provided by placing a first excision construct and a first repression construct (EC₁ and RC₁) in different non-allelic chromosomes and by linking a second repression construct (RC₂) to said first excision construct (EC₁) and repressing a second excision construct (EC₂) linked to said first repression construct (RC₁) with said second repression construct (RC₂), which is linked to the transgene of interest (TGI) and said first excision construct (EC₁).

34. The method according to claim 1, wherein the repression is provided by a double repressible excision system (RES) having two excision constructs (ECs).

35. The method according to claim 1, wherein a triple repressible excision system (RES) is provided by three transgenic constructs/inserts are placed in different non-allelic chromosomes.

36. The method according to claim 1, wherein a multiple repressible excision system (RES) provided by more than three repression constructs (RCs) are used.

37. The method according to claim 1, wherein a complex repressible excision system/recoverable block of function (RES/RBF) is provided by combining the excision and repression constructs (ECs and RCs) with constructs used in a recoverable block of function (RBF) system.

38. The method according to claim 37, wherein the complex repressible excision system/recoverable block of function (RES/RBF) comprises the genes encoding barnase and barstar enzymes.

39. A DNA construct or repressible excision system (RES) for controlling transgene segregation and for preventing the escape of a transgene into the environment by a molecular mechanism said DNA construct comprising
(a) one or more transgenes of interest (TGIs) encoding one or more desired gene products;
(b) said transgenes of interest (TGIs) being closely linked to an excision construct (EC) comprising at least one gene encoding a recombinase enzyme, which is operably linked to
(c) one or more regulating sequences or repression constructs (RCs) comprising regulating sequences which are capable of controlling the expression and/or repression of the recombinase enzyme as a response to at least one externally applicable means alone or in combination with one or more separate repression constructs (RCs);
(d) said excision constructs (ECs) and transgenes of interest (TGIs) and optionally one or more repression construct (RCs) forming a transgenic insert flanked by excision recognition sites (ERSs);
(e) optionally one or more repression constructs (RCs) comprising regulating sequences placed in the same or different chromosomes.

40. The DNA construct according to claim 39, wherein the excision construct (EC) comprises a gene encoding the recombinase enzyme, which is expressed constitutively or in an organ or development specific manner.

41. The DNA construct according to claim 39, wherein the excision construct (EC) comprises a regulating sequence or a promoter controlling the constitutive or organ or development specific expression of the gene encoding the recombinase enzyme.

42. The DNA construct according to claim 41, wherein the regulating sequence(s) controlling the constitutive expression of the gene encoding recombinase enzyme is a CaMV 35S or a nopaline synthase (NOS) promoter.

43. The DNA construct according to claim 39, wherein the regulating sequences controlling the expression of the gene encoding the recombinase enzyme in organ or development specific manner is cysteine endopeptidase (SH-EP) promoter.

44. The DNA construct according to claim 39, wherein the repression construct (RC) comprises a regulating sequence the expression of which controls the repression of the expression of the gene encoding the recombinase enzyme in a constitutive or inducible manner.

45. The DNA construct according to claim 44, wherein the regulating sequence(s) is a 35S promoter from CaMV.

46. The DNA construct according to claim 44, wherein the regulating sequence(s) is a nopaline synthase (NOS) promoter.

47. The DNA construct according to claim 44, wherein the regulating sequence(s) is an inducible heat shock (HS) promoter.

48. The DNA construct according to the claim 47, wherein the regulating sequence is a heat shock (HS) promoter induced by rising the temperature.

49. The DNA construct according to claim 44, wherein the regulating sequence(s) is a Tn10 tet operator-repressor system.

50. The DNA construct according to the claim 49, wherein the regulating sequence is a Tn10 tet operator-repressor system inducible by tetracycline.

51. The DNA construct according to claim 39, wherein the repression construct (RC) comprises regulating sequences the expression of which represses the gene encoding the recombinase enzyme continuously.

52. The DNA construct according to claim 39, wherein the repression construct (RC) comprises nucleotide sequences the expression of which represses the gene encoding the recombinase enzyme and which is optionally inducible as a response to externally applicable means.

53. The DNA construct according to the claim 39, wherein the repression construct (RC) comprises
(a) a nucleotide (operator) sequence(s) capable of blocking the promoter;
(b) a gene expressing antisense RNA of the recombinase enzyme; or
(c) a gene expressing a binding protein of the operator sequence(s) in the promoter of the recombinase enzyme.

54. The DNA construct according to the claim 53, wherein the repression construct (RC) comprises a nucleotide sequence encoding a gene producing a substance, which is capable of blocking the promoter sequence in the excision construct (EC).

55. The DNA construct according to the claim 53, wherein the block of the promoter comprises an operator sequence introduced into the promoter of the gene encoding a recombinase enzyme.

56. The DNA construct according to the claim 53, wherein the repressor construct (RC) expresses a protein having the ability to bind to the operator sequence introduced into the promoter of the gene encoding for the recombinase enzyme, and thereby to repress the excision construct (EC).

57. The DNA construct according to the claim 39, wherein the gene present in the excision construct (EC) and which encodes a recombinase enzyme comprises the cre recombinase gene and the repression construct (RC) comprises a Tn10 tet repressor—operator system.

58. The DNA construct according to the claim 53, wherein the repression construct (RC) comprises a gene expressing antisense RNA of the gene encoding the recombinase enzyme.

59. The DNA construct according to the claim 53, wherein the nucleotide sequence of the repressor construct (RC) encodes an antisense cre recombinase RNA, which expression launches a silencing mechanism of cre recombinase expression.

60. The DNA construct according to claim 39, wherein the gene encoding the recombinase enzyme is placed between two transgenes of interest (TGI)

61. The DNA construct according to claim 39, wherein the gene encoding the recombinase enzyme is placed in an intron of the transgene of interest (TGI).

62. The DNA construct according to claim 39, wherein the excision and repression constructs (EC and RC) are placed in a complex system for controlling the escape of transgene of interest (TGI) said complex system comprising one or more excision and repression constructs (ECs and RCs) of the repressable excision system (RES) optionally combined with an optional number of constructs of a recoverable block of function (RBF) system.

63. The DNA construct according to claim 39, wherein the complex system for controlling the escape of an transgenic insert is a reversed, delayed, double, triple, reverse-delayed or multiple RES-system.

64. The DNA construct according to claim 39, wherein a general repressible excision system (RES) comprises excision and repression constructs (EC and RC) placed in the same chromosomes.

65. The DNA construct according to claim 39, wherein a delayed repressible excision system (RES) comprises excision and repression constructs (EC and RC) located in different non-allelic chromosomes.

66. The DNA construct according to claim 39, wherein a reversed delayed repressible excision system (RD-RES) comprises a first excision construct and a first repression construct (EC₁ and RC₁) located in different non-allelic chromosomes and a second repression construct (RC₂) linked to said first excision construct (EC₁), and said second excision construct (EC₂) linked to said first repression construct (RC₁), which is repressed with said second repression construct (RC₂) linked to the transgene of interest (TGI) and said first excision construct (EC₁).

67. The DNA construct according to claim 39, wherein a double repressible excision system (RES) comprises two repression constructs (RCs).

68. The DNA construct according to claim 39, wherein a multiple repressible excision system (RES) comprises three transgenic inserts introduced into different non-allelic chromosomes.

69. The DNA construct according to claim 39, wherein a multiple repressible excision system (RES) comprises more than three repression constructs (RCs).

70. The DNA construct according to claim 39, wherein a complex repressible excision system (RES) comprises excision and repression constructs (ECs and RCs) combined with recoverable block of function (RBF) constructs.

71. The DNA construct according to claim 70, wherein the complex repressible excision system (RES) comprises the excision and repression constructs (ECs and RCs) combined with the genes encoding barnase and barstar enzymes.
